(19) United States
(12) Patent Application Publication
(10) Pub. No.: US 2009/0248090 A1
(43) Pub. Date: Oct. 1, 2009

(54) RIB BONE TISSUE CLAMP

(75) Inventors: Charles Gordon, Tyler, TX (US); James E. Deaton, Georgetown, TX (US)

Correspondence Address: FULBRIGHT & JAWORSKI L.L.P., 600 CONGRESS AVE., SUITE 2400, AUSTIN, TX, 78701, US

(73) Assignee: PRONTO PRODUCTS, LLC, Tyler, TX (US)

(21) Appl. No.: 12/342,777

(22) Filed: Dec. 23, 2008

Related U.S. Application Data
(60) Provisional application No. 61/017,336, filed on Dec. 28, 2007.
(60) Provisional application No. 61/023,327, filed on Jan. 24, 2008.
(60) Provisional application No. 61/104,199, filed on Oct. 9, 2008.
(60) Provisional application No. 61/108,417, filed on Oct. 24, 2008.

(51) Int. Cl. A61B 17/84

(52) U.S. Cl. 606/324

(57) ABSTRACT

Systems, methods, and kits incorporating a clamp for securing to rib bone tissue.

### RELATED APPLICATIONS

This application claims priority to, and incorporates by reference, each of the following applications: U.S. Provisional Patent Application 61/017,336 filed Dec. 28, 2007; U.S. Provisional Patent Application 61/023,327 filed Jan. 24, 2008; U.S. Provisional Patent Application 61/104,199 filed Oct. 9, 2008; and U.S. Provisional Patent Application 61/108417 filed Oct. 24, 2008.

### BACKGROUND INFORMATION

### Field of the Invention

Exemplary embodiments of the present disclosure comprise a devices that can be secured to bone tissue in ribs and methods of securing the devices.

Exemplary embodiments comprise features that provide for ease of installation of the clamp onto a rib bone. In addition, exemplary embodiments comprise features that assist in securing the clamp to the rib bone.

### SUMMARY

Exemplary embodiments of the present disclosure provide novel systems, kits, and methods for securing medical devices to bone tissue in rib bones.

In certain embodiments, a clamp may comprise gripping members that comprise projections directed toward the open space. The clamp may be configured to allow a foreign object to be inserted into the open space. The clamp may be configured to exert a compressive force on the foreign object in certain embodiments, and a cross-section of the first clamp may be generally U-shaped. The first side and the second side of a clamp may be comprised of a unitary piece. In particular embodiments, the first side comprises a first end portion distal from the coupling portion; the second side comprises a second end portion distal from the coupling portion; and the first side and the second side are angled towards each other so that the first end portion is proximal to the second end portion.

Particular embodiments comprise a bone tissue clamp comprising: a first side; a second side; a coupling portion coupling the first side to the second side; an open space between the first and second side; and a plurality of gripping members disposed on at least one of the first and second side, wherein the bone tissue clamp is configured for securement to a rib. In certain embodiments, the gripping members comprise crimped portions of the bone tissue clamp. In specific embodiments, the gripping members comprise a plurality of teeth. In particular embodiments, the first side and the second side are angled toward each other when the bone tissue clamp is viewed from a first end of the bone tissue clamp.

In specific embodiments, the coupling portion comprises a first aperture. In particular embodiments, the coupling portion comprises a second aperture. The first aperture and the second aperture can be configured to receive a tool that can be manipulated to spread apart the first side and the second side. In certain embodiments, the gripping members are distal from the coupling portion. The aperture can also be configured to allow access to a rib when the clamp is installed on the rib.

In specific embodiments, the first and second sides each comprise an extended central portion. In particular embodiments, the extended central portions of the first and second sides comprise a plurality of gripping members. The first side may comprise a pocket that extends away from the first side. In certain embodiments, the first and second side each comprise a central portion that is shorter than the remainder of the first and second sides.

Specific embodiments also comprise a method of securing a fracture of a rib. The method may comprise: providing a bone tissue clamp according to any of clamps described above, and securing the bone tissue clamp to the rib so that the first side of the bone tissue clamp is engaged with a first side of the rib and a second side of the bone tissue clamp is secured to a second side of the rib.

### BRIEF DESCRIPTION OF THE FIGURES

While exemplary embodiments of the present invention have been shown and described in detail below, it will be clear to the person skilled in the art that changes and modifications may be made without departing from the scope of the invention. As such, that which is set forth in the following description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the invention described herein can be included within the scope of the present invention. For example, different materials of construction may be used for the clamps or coupling members employed in the kit or system. Furthermore, the shape of individual clamps or coupling members may also be altered.

In the following Detailed Description of Disclosed Embodiments, various features are grouped together in several embodiments for the purpose of streamlining the disclosure. This method of disclosure is not to be interpreted as reflecting an intention that exemplary embodiments of the invention require more features than are expressly recited in each claim. Rather, as the following claims reflect, inventive subject matter lies in less than all features of a single disclosed embodiment. Thus, the following claims are hereby incorporated into the Detailed Description of Exemplary Embodiments, with each claim standing on its own as a separate embodiment.

Identical reference numerals do not necessarily indicate an identical structure. Rather, the same reference numeral may be used to indicate a similar feature or a feature with similar functionality. Not every feature of each embodiment is labeled in every figure in which that embodiment appears, in order to keep the figures clear. Similar reference numbers (e.g., those that are identical except for the first numeral) are used to indicate similar features in different embodiments.

FIG. 1 is a side view of a clamp installed on a rib according to an exemplary embodiment of the present disclosure.

FIG. 2 is a perspective view of a clamp on a rib bone according to an exemplary embodiment of the present disclosure.

FIG. 3 is a perspective view of the clamp of FIG. 2.

FIG. 4 is perspective view of a clamp according to an exemplary embodiment of the present disclosure.

FIGS. 5 and 6 are perspective views of a clamp according to an exemplary embodiment of the present disclosure.

FIGS. 7 and 8 are perspective views of a clamp according to an exemplary embodiment of the present disclosure.

### DETAILED DESCRIPTION OF EXEMPLARY EMBODIMENTS

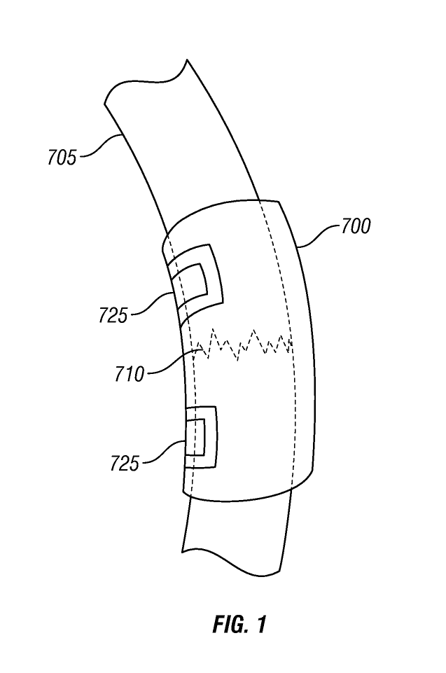

In an exemplary embodiment shown in FIG. 1, a clamp 700 may be used to secure a fractured rib 705. In this embodiment clamp 700 is placed on rib 705 so that it is secured to each side of a fracture 710 in rib 705. Gripping members 725 and 720 grip rib 705 on each side of fracture 710. In this embodiment, gripping members 725 are crimped areas, but in other embodiments gripping members may be a different configuration.

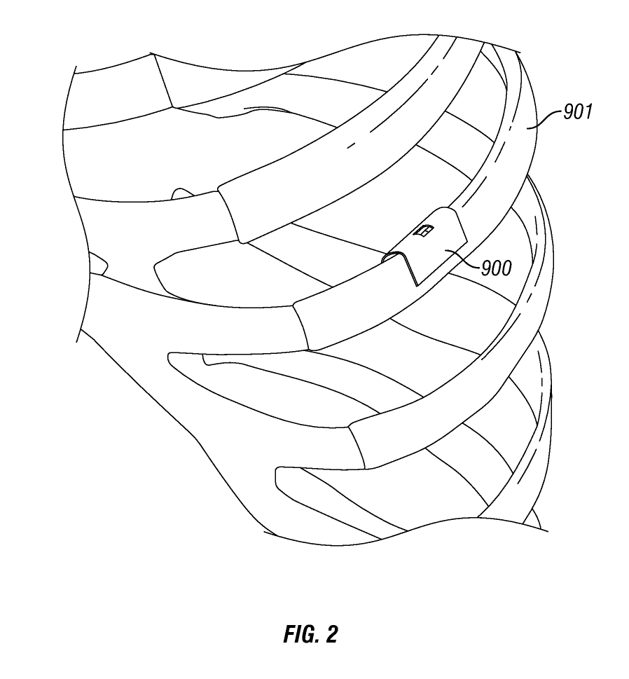

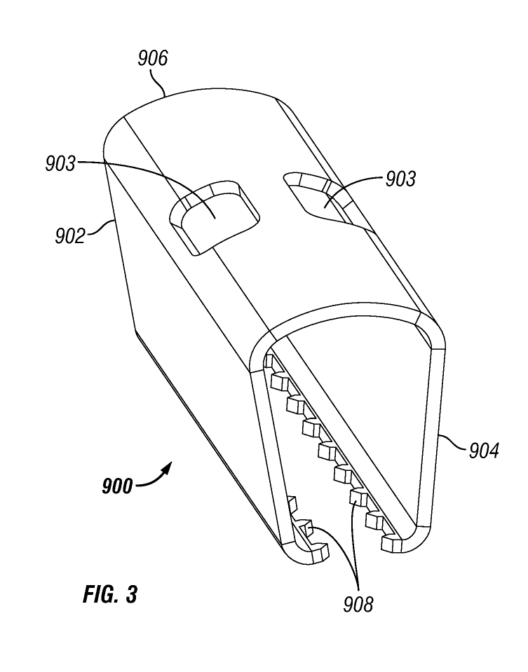

Referring now to FIGS. 2 and 3, a clamp 900 may be used as a rib fixation device. Clamp 900 comprises a coupling portion 906 that couples a first side 902 to a second side 904. In the embodiment shown, first and second sides 902, 904 each comprise gripping members 908 at an end distal from coupling portion 906. In the embodiment shown, gripping members 908 comprise a series of teeth, but other embodiments may comprise gripping members with different configurations.

Similar to previously-described embodiments, first and second sides 902, 904 may be angled toward each other so that they provide a compressive force on rib bone 901 and aid in securing clamp 900 to bone 901. In the embodiment shown, coupling portion 906 may also comprise a pair of apertures 903 that allow a tool (not shown) to be inserted for spreading first and second sides 902 and 904 away from each other. The use of a tool to spread clamp 900 farther open can make it easier to install. In addition, apertures 903 may be used to provide access to the bone underneath clamp 900.

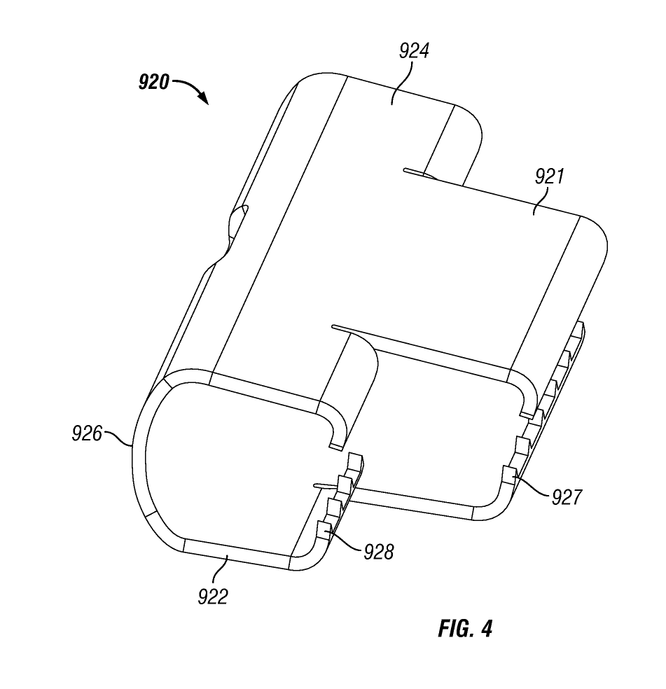

Another alternative embodiment of a rib fixation device or clamp is shown in FIG. 4. As shown in FIG. 4, clamp 920 is generally equivalent to clamp 900 described above and comprises a coupling portion 926 between first and second sides 922, 924 that comprise gripping members 928. However, clamp 920 may comprise an extended central portion 921 of first and second sides 922 and 924. Central portion 921 may comprise gripping members 927 that are not collinear with gripping members 928. Such a configuration may be beneficial if it is desired to secure rib bone 901 in different locations (for example depending on the shape of the fracture).

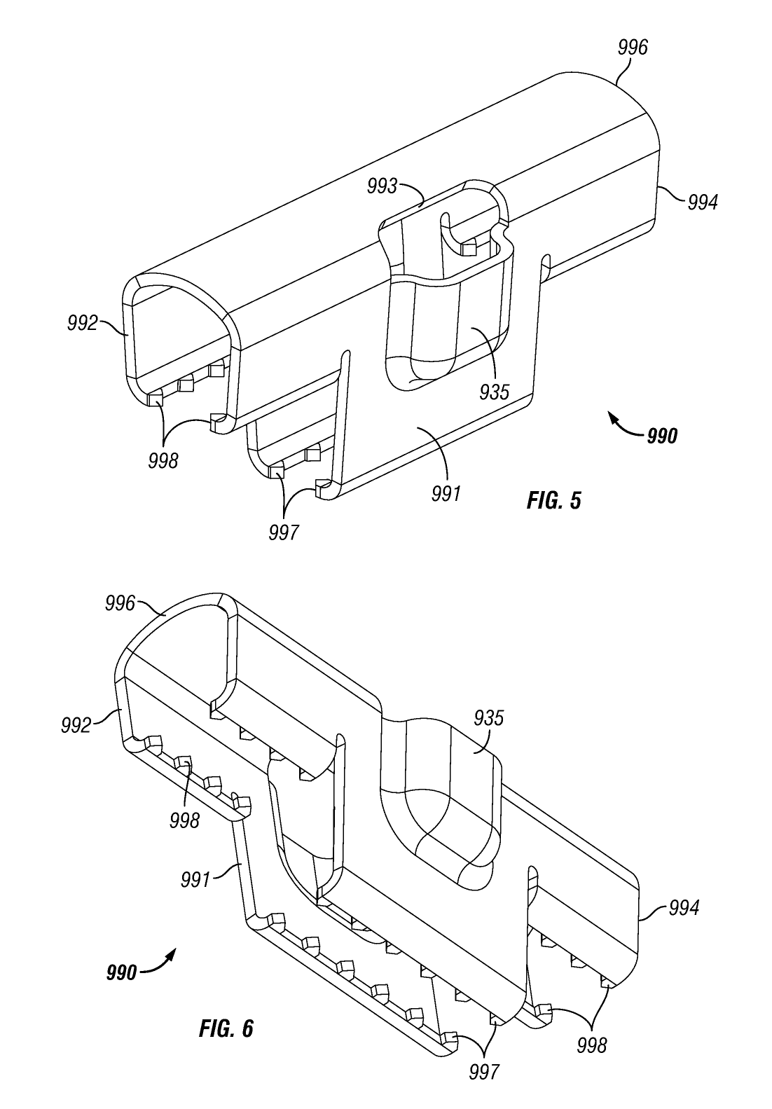

Another exemplary embodiment of a rib clamp 990 is shown in FIGS. 5 and 6. Clamp 990 is similar to the embodiment shown in FIG. 4, but also comprises a pair of pockets 935 proximal to apertures 993. The additional features of clamp 990 labeled with “99X” are equivalent to the features labeled in the embodiment of FIG. 4 with “92X”. The pockets 935 may assist in engaging an installation tool (not shown) used to spread first and second sides 992 and 994.

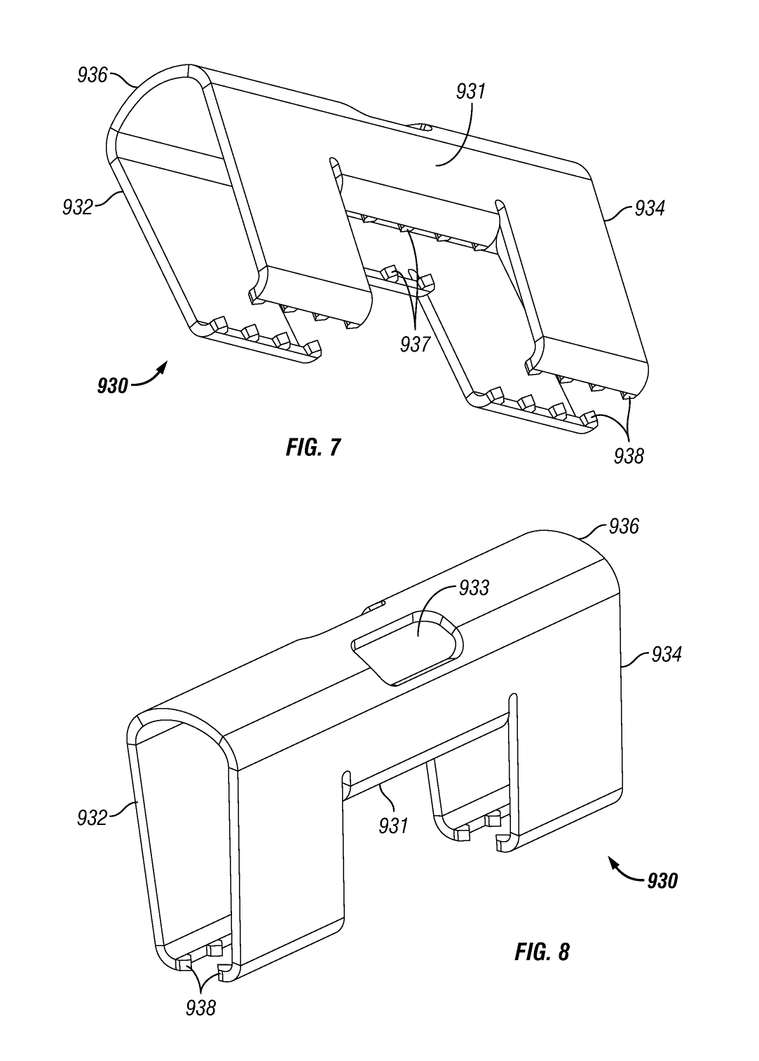

Yet another exemplary embodiment of a rib bone clamp 930 is shown in FIGS. 7 and 8. This embodiment is similar to the embodiment of FIG. 4, with the exception that central portion 931 is shorter, instead of longer, than the remainder of first and second sides 932 and 934. Clamp 930 comprises sides 932 and 934, and a coupling portion 936 with apertures 933 similar to previously-described embodiments. In addition, central portion 931 comprises gripping members 937 that are not collinear with gripping members 938.

In certain exemplary embodiments, the tools used to set the clamps can be sterilized and re-used. In other exemplary embodiments, the tools used to set the clamps will be designed for one-time use. Clamps according to exemplary embodiments may be manufactured from suitable medical-grade materials, including titanium and stainless steel.

It should be understood that the present system, kits, apparatuses and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description of Exemplary Embodiments, various features are grouped together in several embodiments for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description of Exemplary Embodiments, with each claim standing on its own as a separate embodiment.

1. A bone tissue clamp comprising:
a first side;
a second side;
a coupling portion coupling the first side to the second side;
an open space between the first and second side; and
a plurality of gripping members disposed on at least one of the first and second side, wherein the bone tissue clamp is configured for securement to a rib, wherein the gripping members comprise crimped portions of the bone tissue clamp.

2. The bone tissue clamp of claim 1 wherein the gripping members comprise a plurality of teeth.

3. The bone tissue clamp of claim 1 wherein the first side and the second side are angled toward each other when the bone tissue clamp is viewed from a first end of the bone tissue clamp.

4. The bone tissue clamp of claim 1 wherein the coupling portion comprises a first aperture.

5. The bone tissue clamp of claim 4 wherein the coupling portion comprises a second aperture.

6. The bone tissue clamp of claim 5 wherein the first aperture and the second aperture are configured to receive a tool that can be manipulated to spread apart the first side and the second side.

7. The bone tissue clamp of claim 1 wherein the gripping members are distal from the coupling portion.

8. The bone tissue clamp of claim 1 wherein the aperture is configured to allow access to a rib when the clamp is installed on the rib.

9. The bone tissue clamp of claim 1 wherein the first and second sides each comprise an extended central portion.

10. The bone tissue clamp of claim 1 wherein the extended central portions of the first and second sides comprise a plurality of gripping members.

11. The bone tissue clamp of claim 1 wherein the first side comprises a pocket that extends away from the first side.

12. The bone tissue clamp of claim 1 wherein the first and second side each comprise a central portion that is shorter than the remainder of the first and second sides.

13. A method of securing a fracture of a rib, the method comprising:
providing a bone tissue clamp according to any of claims 1-12; and
securing the bone tissue clamp to the rib so that the first side of the bone tissue clamp is engaged with a first side of the rib and a second side of the bone tissue clamp is secured to a second side of the rib.
